SEQUENCE LISTING (4 sequences)

SEQ ID NO: 1 - DNA, 20 bp, Artificial
  PCR Primer
cccgaactggaatcttcctt

SEQ ID NO: 2 - DNA, 20 bp, Artificial
  PCR Primer
gggtttgccctctttttctc

SEQ ID NO: 3 - protein, 31 aa, Homo sapiens
VLENSTKNSNDPAVFKDNPTEDVEYQCVADN

SEQ ID NO: 4 - protein, 31 aa, Artificial
  Nonsense peptide with no known matches.
TEDVLVPQNKSDNKATNNAFPNVSYVDEEDC

(19) United States
(12) Patent Application Publication
(10) Pub. No.: US 2009/0215171 A1
(43) Pub. Date: Aug. 27, 2009

(54) PELCAM-1-related molecules compositions kit of parts and associated methods of use

(75) Inventors: Subroto Chatterjee, Columbia, MD (US); Heming Wei, Singapore (SG)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/317,096

(22) Filed: Dec. 19, 2008

Related U.S. Application Data
(63) Continuation of application No. 11/158,447, filed on Jun. 21, 2005, now abandoned.
(60) Provisional application No. 60/581,985, filed on Jun. 21, 2004.

(51) Int. Cl. C12N 5/08

(52) U.S. Cl. 435/366

(57) ABSTRACT

An isolated polynucleotide coding for platelet endothelial cell adhesion molecule-1 (PECAM-1), and obtainable by amplifying cDNA from total human white blood cells by PCR; peptides encoding PECAM-1 5^{th} Ig-like domain; an antibody against one of the peptides; associated compositions methods and kits of parts.

### RELATED APPLICATIONS

This application claims priority to U.S. provisional No. 60/581,985 application entitled “Pecam-1 Related Composition And Associate Method Of Use” filed on Jun. 20, 2004 attorney docket number “70292-0010600, herein incorporated by reference in its entirety.”

### BACKGROUND OF THE DISCLOSURE

Platelet endothelial cell adhesion molecule-1 (PECAM-1) is an integral protein in vascular endothelial cells (EC) [1-4] and is implicated in the integrin-mediated cell adhesion[5, 6], TEM of monocytes/leukocytes [4, 7] as well as apoptosis [8, 9]. Studies up-to-date implicate the major role of extracellular (Ig)-like domains of PECAM-1, mainly the 1^{st} and 2^{nd} domains, in mediating homophilic binding and leukocyte diapedesis [2, 7, 10-16]. Use of antibodies against the 1^{st} and 2^{nd} extracellular immunoglobulin (Ig)-like domains has consistently shown inhibitory effects on leukocyte TEM [7, 13, 15, 17]. On the other hand, soluble chimeras made of the entire extracellular portion of PECAM-1 or of only the first (Ig)-like domain of PECAM-1, fused to the Fc portion of IgG, have been shown to block diapedesis in vitro and in vivo[7, 17].

Another essential feature of the extracellular (Ig)-like domains of PECAM-1, besides mediating cell adhesion and TEM, is its involvement in intracellular Ca²⁺ homeostasis. PECAM-1 has multiple cation-binding sites on the 5^{th} (consists of 2 binding sites) and 6^{th} (consists of 3 binding sites) extracellular (Ig)-like domains [18], and the 6^{th} (Ig)-like domain has been shown to play a major role in maintaining intracellular Ca²⁺ homeostasis: Antibody against the 6^{th} (Ig)-like domain could activate plasmalemmal Ca²⁺-conducting channels and trigger the increase of intracellular free [Ca²⁺] in EC [19, 20] while antibodies against the 1^{st} and 2^{nd} (Ig)-like domains have only weak effect on intracellular [Ca²⁺]_{i [}19]. The engagement of soluble recombinant PECAM-1 consisting of the 1^{st} and 2^{nd} domains could also increase [Ca²⁺]_{i [}20]. More recent studies reveal that PECAM-1 may participate in signaling via tyrosine phosphorylation with its immunoreceptor tyrosine-based inhibitory motif (ITIMs) at its cytoplasmic domains and form PECAM-1/cytoskeleton interaction with β and γ catenins [21-23].

Structural variations in PECAM-1 gene and protein products known up-to-date are several PECAM-1 isoforms reported in both human and rodent due to alternative splicing [24-27]. Those isoforms involve only small exon(s) in the cytoplasmic domains of PECAM-1 and no isoform involve the extracellular domain(s) of PECAM-1 have been reported so far.

### SUMMARY OF THE DISCLOSURE

According to a first aspect, an isolated polynucleotide is disclosed, the polynucleotide coding for platelet endothelial cell adhesion molecule-1 (PECAM-1) herein also referred as Δ exon7, the polynucleotide having the size of about 375 bp, the polynucleotide obtainable by amplifying cDNA from total human white blood cells by PCR using as primers a first oligonucleotide comprising sequence SEQ ID NO: 1 and a second oligonucleotide comprising the sequence SEQ ID NO: 2.

According to a second aspect, in a method for testing the biological function of the 5^{th} Ig-like extracellular domain of PECAM-1, an improvement is disclosed. The improvement comprises providing an isolated polynucleotide coding for a platelet endothelial cell adhesion molecule-1, the polynucleotide having the size of about 375 bp, the polynucleotide obtainable by amplifying cDNA from total human white blood cells by PCR using as primers a first oligonucleotide comprising sequence SEQ ID NO: 1 and a second oligonucleotide comprising the sequence SEQ ID NO: 2, a cell expressing said isolated polynucleotide or a composition comprising said polynucleotide together with suitable vehicle carrier or auxiliary agents.

According to a third aspect a first peptide is disclosed, the peptide comprising the amino acid sequence SEQ ID NO: 3.

According to a fourth aspect a second peptide is disclosed, the second peptide comprising the amino acid sequence SEQ ID NO: 4

According to a fifth aspect, an antibody is disclosed, the antibody, bonding to an isolated peptide having the amino acid sequence of SEQ ID NO: 3.

According to a sixth aspect a method to regulate intracellular calcium homeostasis of a human cell, is disclosed, the method comprising administering to the human cell an effective amount of a peptide having amino acid sequence of SEQ ID NO: 3 and/or an effective amount of an antibody which bonds to an isolated peptide of SEQ ID NO: 3.

According to a seventh aspect a method for inhibiting PECAM-1 dependent trans-endothelial migration (TEM) of a monocyte is disclosed, the method comprising administering to a monocyte an effective amount of a peptide having amino acid sequence of SEQ ID NO: 3 and/or an effective amount of an antibody which bonds to an isolated peptide of SEQ ID NO: 3.

According to a eight aspect, a kit of parts for the regulation of intracellular calcium homeostasis of a human cell is disclosed, the kit comprising a peptide comprising the sequence SEQ ID NO: 3; and an antibody which bonds to an isolated peptide having the amino acid sequence of SEQ ID NO: 3, the peptide and the antibody to be administered in an effective amount to the human cell thereby regulating the intracellular calcium homeostasis of the human cell.

The kit of part, can further comprise a second peptide comprising the sequence SEQ ID NO:4 the second peptide to be administered in combination with the first peptide and the antibody as a negative control wherein administration of the effective amount of the first peptide and the antibody regulate the intracellular calcium homeostasis of the human cell.

According to a ninth aspect, a kit of parts for inhibiting PECAM-1 dependent trans-endothelial migration of a monocyte is disclosed, the kit comprising a first peptide comprising the sequence SEQ ID NO: 3; and an antibody which bonds to an isolated peptide having the amino acid sequence of SEQ ID NO: 3, the first peptide and the antibody to be administered in an effective amount to a monocyte thereby inhibiting the PECAM-1 dependent TEM of the monocyte.

The kit of part, can further comprise a second peptide comprising the sequence SEQ ID NO:4 the second peptide to be administered in combination with the first peptide and the antibody as a negative control wherein administration of the effective amount of the first peptide and the antibody inhibit the PECAM-1 dependent TEM of the monocyte.

According to further aspects compositions comprising at least one of the first peptide, the second peptide and the antibody together with a suitable carrier vehicle or auxiliary agent are disclosed. Further details concerning the identification of the suitable carrier vehicle or auxiliary agent of the compositions, and generally manufacturing and packaging of the kit, can be identified by the person skilled in the art upon reading of the present disclosure.

A person skilled in the art can identify modalities, dosages, timing of administration of the methods herein disclosed as well as vehicle carrier auxiliary agents, relative concentration, formulation and modalities of administration of the compositions herein disclosed.

As used herein, the term “antibody” may be a polyclonal or monoclonal antibody unless differently specified. The relevant preparation, is identifiable by a person skilled in the art upon reading of the present disclosure. Antibody fragments, which retain the ability to recognize the antigen of interest, are included as well.

The polynucleotides, peptides, methods compositions and kits of the disclosure will be exemplified with the aid of the enclosed figures.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 illustrates the identification of a PECAM-1 transcript missing the entire 7^{th} exon of human PECAM-1 gene; RT-PCR was performed with total RNA from blood mononuclear cell derived from 4 human volunteers. A: image of the PCR product resolved on a 1.5% agarose gel. The expected PCR product was 651 base pairs. A lower band with the size of 376 base pairs also emerged. B: Southern blotting image of the above-mentioned PCR products that were transferred to a nylon membrane and hybridized with a radioactive labeled PECAM-1 cDNA probe. C: Sequences of TA cloned upper and lower PCR bands showing a deletion of entire Exon 7 in the lower band (a) in comparison to the full-length sequence from the upper band (b);

FIG. 2 illustrates the characterization of JHS-7 Ab-A, JHS-7 Ab recognized PECAM-1 in HUVEC lysate. A total of 25 μg of HUVEC lysate was run on a 12% polyacrylamide gel along with a soluble recombinant PECAM-1 protein (95-98 KDa) containing only the extracellular domains of PECAM-1. There identical blots were prepared and hybridized sequentially with (a) JHS-7 Ab; (b) mAb-N (a monoclonal Ab raised against extracellular domains of PECAM-1); and (c) pAb-C (a polyclonal Ab raised against the cytoplasmic domains of PECAM-1). B. JHS-7 Ab recognized native membrane-bound PECAM-1 in (a) HUVECs, (b) L-cell stablely transfected with human PECAM-1, and (c) U-937 cells.

FIG. 3 illustrates the effects of JHS-7 Ab on monocyte TEM; Confluent monolayers of HUVEC grown on the top wells of a Transwell® plate and the U-937 cells migrated to the bottom wells were counted and bar graphs plotted. HUVECs were incubated with (A): JHS-7 Ab at 0.4, 0.8 μg/mL and 1.6 μg/mL; a control anti-PECAM-1 antibody pAb-C (a polyclonal Ab raised against the cytoplasmic domains of PECAM-1) at 1.6 μg/mL; and rabbit IgG at 1.6 μg/mL. (B): Effects pf JHS-7 peptide on the trans-endothelial migrating freshly prepared human monocytes. The data represents average values from three experiments analyzed in quadruplicate.

FIG. 4 illustrates the effects of JHS-7 peptide on monocyte TEM; Confluent monolayers of HUVEC grown on the top wells of a Transwell® plate and the U-937 cells migrated to the bottom wells were counted and bar graphs plotted. HUVECs were incubated with (A): JHS-7 peptide and a scrambled peptide (Sc) both at 15 and 30 μg/m. All incubations were carried out for 16 hours. Next, pre-stained U-937 cells were applied on top of the HUVEC monolayers followed by 8-hours transmigration period. (B) Effects pf JHS-7 peptide on the trans-endothelial migrating freshly prepared human monocytes; the data represents average values from three experiments analyzed in quadruplicate.

FIG. 5 illustrates the effects of JHS-7 Ab and Peptide on Ca²⁺ mobilization; [Ca²⁺]ᵢ was measured in HUVEC suspension and the release Ca²⁺ from intracellular stores (the peak ca²⁺) was induced by extra-cellular thrombin. (A): a—JHS-7 Ab at 1.6 μg/mL, b—JHS-7 Ab at 0.4 μg/mL, c—pAb-C at 1.6 μg/mL, d and e—HUVECs pre-exposed to TNF-α (5 ng/mL for 4 hours) followed by JHS-7 Ab at 1.6 and 0.4 μg/mL respectively. (B): a—JHS-7 peptide at 30 μg/mL, b—JHS-7 peptide at 15 μg/mL, c—a scrambled peptide 30 μg/mL, and d—JHS-7 Peptide at 30 μg/mL, in addition, EGTA (3 mM) was added in the cuvette prior to thrombin stimulation; following thrombin induced Ca²⁺ peak, Ca²⁺ (3 mM) was added in the cuvette to restore the baseline Ca²⁺ level; data represents average values from three experiments analyzed in triplicates.

### DETAILED DESCRIPTION OF THE PREFERRED EMBODIMENTS

The present disclosure includes several aspects. First, the inventors have identified a novel PECAM-1 transcript in the region of extracellular domains (missing the entire 7^{th} exon) in human subjects; second, employing a synthetic peptide (JHS-& peptide) encoding the 5^{th} (Ig)-like domain and corresponding antibody (JHS-7 Ab) against the peptide, the Applicants have demonstrated attenuated monocyte trans-endothelial migration through the monolayers of HUVECs (rest and TNF-α activated) treated with both JHS-Ab and peptide; third, both the JHS-7 Ab and peptide were involved in regulating intracellular calcium homeostasis, and this may help to explain the mechanism of the inhibitory effect of JHS-7 Ab and peptide on monocyte diapedesis.

PECAM-1 is a major constitutively expressed protein in mammalian endothelial cells, platelets and monocytes. Up to now the fully length PECAM-1 protein (130 kDa) and a soluble PECAM-1 (110 kDa) have been detected in human cells [1, 30], and plasma respectively [30, 31]. In both human and rodents, several PECAM-1 transcripts and isoforms missing one or two of the small exons (exon 11-15) at the 3′ end of PECAM-1 due to alternative splicing, have been reported in a variety of tissue and cells including hematopoietic cells [24-27].

However the transcripts and isoforms are all limited in the cytoplasmic domains, no alternative transcripts involved the extracellular domain has been reports so far. The Applicants discovered a novel PECAM-1 transcript missing the entire exon 7 that encodes the 5^{th} extracellular (Ig)-like domain. Such a transcript is most likely due to alternative splicing during gene transcription in haemostatic cells as it was undetectable in HUVECs (from >5 HUVEC cell pellets, data not shown). In the following studies, this PECAM-1 transcript was also detected in peripheral mononuclear cells and platelets from more healthy volunteers and patients with severe atherosclerosis. Up-to-date, the Applicants have confirmed that quantitative individual variations, however, the Applicants' data is insufficient to suggest any correlations with phenotypic changes.

The alternative transcript (Δ exon7) encodes the 5^{th} extracellular (Ig)-like domain and could encode a protein isoform lacking the 5^{th} extracellular (Ig)-like domain. This polynucleotide can be used in methods for testing a biological function of the 5^{th} Ig-like extracellular domain PECAM-1, wherein the methods and modalities can be identified by a person skilled in the art.

Previous studies have assigned the functional roles of the 1^{st} and 2^{nd} extracellular (Ig)-like domains of PECAM-1 with leukocyte adhesion and TEM and the role of the 6^{th} extracellular (Ig)-like domain with intracellular Ca²⁺ regulation both in vitro and in vivo [7, 16, 32-35]. The experiments were conducted with either antibodies or recombinant/chimera soluble proteins or peptides. Up to date, little knowledge, if any, is available on the role of 5^{th} (Ig)-like extracellular domain of PECAM-1 [13, 18].

Following those findings, the Applicants initiated studies on the functional role of the 5^{th} domain following a similar strategy by employing a JHS-7 Ab and peptide. The Applicants' studies suggested that JHS-Ab and JHS-7 peptide are capable of inhibiting PECAM-1 dependent monocytes TEM. The JHS-7 peptide prepared in the Applicants' studies was equally or more effective in abrogating up to 80% of TEM of monocytes and this is in the same order as published previously employing the entire extracellular domain of PECAM-1 [10]. Further studies are required to determine the efficacy of this peptide in abrogating TEM in vivo.

The inhibitory effects of JHS-7 Ab and peptide on monocyte TEM could be attributed to the alteration of intracellular Ca²⁺ homeostasis in HUVEC's. In endothelial cells the intracellular Ca²⁺ release following monocyte adhesion is required for the transendothelial migration of monocytes [36].

The extracellular (Ig)-like domains of PECAM-1 have been associated with intracellular Ca²⁺ in EC. Increased intracellular free Ca²⁺ at baseline level, was observed in EC engaged with soluble recombinant PECAM-1 consisting of the 1^{st} and 2^{nd} domains [19]. Increased intracellular free Ca²⁺ was also detected in a PECAM-1 transfected endothelial-like cell line, but not the untransfected cell line, after engaging with an anti-PECAM-1 mAb, 4G6 (raised against the 6^{th} (Ig)-like domain)[19]. While similar, but less significant effect (on increasing free [Ca²⁺]ᵢ) was observed with the use of mAbs against the 1^{st} and 2^{nd} (Ig)-like domain. In contrast, no effect was observed with control IgG [19].

The Applicants investigated intracellular Ca²⁺ homeostasis in rest and TNF-α pre-activated HUVECs subsequently treated with JHS-7 Ab and peptide. Based on the Applicants' observation that extracellular thrombin stimulates mainly the Ca²⁺ release from intracellular stores in HUVECs, the Applicants' result thus suggested that effects of JHS-7 Ab and peptide are to deplete intracellular stores Ca²⁺. This change might be associated with attenuation of TEM.

The Applicants' additional studies revealed that JHS-7 Ab and peptide did not alter the protein expression of PECAM-1, VCAM-1, ICAM-1, nor the level of PECAM-1 tyrosine phosphorylation (data not shown) in HUVEcs although tyrosine phosphorylation of PECAM-1 has been shown to play a role in platelet aggregation [37].

Embodiments disclosed herein refer to PECAM-1 which is an integral component of endothelial cells (EC) and has been implicated in the trans-endothelial migration (TEM) of circulating leukocytes mediated by its 1st and 2nd extracellular immunoglobulin (Ig)-like domains and regulation of intracellular Ca²⁺ homeostasis with its 6th domain. Up-to-date, little is known about the role of the 5th extracellular (Ig)-like domain. The present disclosure relates to a human PECAM-1 transcript missing the entire 7^{th} exon, which encodes the 5th extracellular (Ig)-like domain of PECAM-1.

To explore the functional role of this domain, a sequence-homology synthetic peptide (JHS-7 peptide) and a corresponding polyclonal antibody (JHS-7 Ab) were prepared and their effects in monocyte TEM and the intracellular Ca²⁺ homeostasis were measured. In resting human umbilical vein EC(HUVECs) and HUVECs pre-activated with tumor necrosis factor-α (TNF-α), both JHS-7 Ab and JHS-7 peptide exerted dose dependent effects on reducing (50˜80%) the TEM of freshly isolated human mononuclear cells and a promonocytic cell line (U-937) accompanied with decreasing the [Ca²⁺]ᵢ in the intracellular Ca²⁺ stores.

Accordingly, a novel PECAM-1 transcript (Δ exon 7) was identified. Additionally the Applicants showed that in EC, the 5^{th} (Ig)-like domain of PECAM-1 can play a role in monocyte TEM and Ca²⁺ homeostasis. In particular, according to this aspect, the Applicants report a novel PECAM-1 transcript missing the entire 7^{th} exon that encodes for the 5^{th} extracellular (Ig)-like domain. Employing a synthetic peptide with sequence homology to the 5^{th} domain and a corresponding polyclonal antibody, the applicants demonstrate that this domain can have a functional role in regulating TEM and Ca²⁺ homeostasis in human endothelial cells.

### Examples

### Materials and Methods

### Cell Culture And Reagents

Primary human umbilical vein endothelial cells (HUVECs) were purchased from Clonetics™, Biowhitttaker (CC-2517-SP) together with specific endothelial culture media (EGM™) including a basal media (CC-3124) and a Bullet Kits® (CC-4133) containing human epithelial growth factor and other supplements. HUVECs were cultured on gelatin (0.2%)-coated surface in EGM™ supplemented with 10% fetal bovine serum (FBS). U-937 cell line, a human pro-monocyte cell line (CRL-1593.2), was purchased from American Type Culture Collection (ATCC), Bethesda, Md. and cultured in RPMI 1640 medium supplemented with 10% FBS. Fresh human peripheral mononuclear cells were isolated from the venous blood of healthy volunteers with Ficoll-Hypaque™ (Amersham Pharmacia Biotech AB, Uppsala, Sweden) following the manufacturer's protocol.

### Antibodies, Reagents and Chemicals

An anti-PECAM-1 monoclonal antibody (mAB) raised against the extracellular domains of PECAM-1, here after refer as “mAb-N”, was purchased from R&D Systems, Minneapolis, Minn. (1 mg/mL, clone 9G11, Cat# BBA7) and used for Western immunoblotting and immunofluorescence assay at 1:500 and 1:250 dilutions, respectively. Another mAb of PECAM-1, here after refer as “mAb-ND1”, was a gift from Dr. Peter Newman and it was raised against the 1^{st} (Ig)-like domain and used as a positive control in the TEM assays. A polyclonal antibody raised against the c-terminal of PECAM-1, here after refer as “pAb-C”, was purchased from Research Diagnostics Inc, Flanders, N.J., USA (Cat# RDI-MCD31cabG, 0.2 ug/mL) and used for Western immunoblotting at a 1:500 dilution. A JHS-7 Ab (0.4 μg/mL) (described later) was used in Western immunoblotting and immunofluorescence assay at 1:500 and 1:50, dilutions respectively. Rabbit IgG (PN31325) was purchased from PIERCE Biotechnology, Rockford, Ill. Soluble recombinant PECAM-1 (with size of 95˜98 KDa, containing only the extracellular domains of PECAM-1) was purchased from R&D Systems, Minneapolis, Minn., USA (Cat# ADP6C). TNF-α (Tumor Necrosis Factor-α (Cat# T-6674), thrombin (Cat# T-7572) and EGTA (Ethylene glycol-bis(2-aminoethylether)-N,N,N′,N′-tetraacetic acid, Cat# E-3889) were from Sigma-Aldrich St Louis, Mo.

RT-PCR. Southern Blot and TA cloning

Total RNA was extracted from fresh mononuclear cells with Trizol Reagent® (Invitrogen life technologies) following the manufacturer's instruction. First strand complementary DNA (cDNA) was synthesized by reverse transcription (RT) with M-MLV-Reverse transcriptase (Invitrogen life technologies) primed with both oligo d(T)₁₅ and random hexamers employing a standard protocol. Polymerase chain reaction (PCR) was carried out to amplify a 651 base pairs cDNA fragment (from position 982 to 1632 of the open reading frame, containing multi-exons). Primers for the PCR were: 5′-CCCGAACTGGAATCTTCCTT-3′ (SEQ ID NO: 1) (forward) and 5′-GGGTTTGCCCTC TTTTTCTC-3′ (SEQ ID NO: 2) (reverse). The PCR was performed by repeating the following amplification cycle for 32 times: denaturing at 94° C. for 45 seconds followed by annealing at 60° C. for 45 seconds and extension at 72° C. for 60 seconds. Southern hybridization was performed on nylon membrane transferred with the above-mentioned PCR products from agarose gel and hybridized with a α-dCTP ³²P labeled PECAM-1 cDNA probe. PCR product was gel-purified and TA cloned with a Promega TA Cloning Kit. Positive clones were picked up and the plasmid DNA was isolated and sequenced.

### Preparation of JHS-7 Peptide and JHS-7 Antibody

An anti-human PECAM-1 polyclonal antibody (against the 5^{th} (Ig)-like extracellular domain), here after referred as “JHS-7 Ab” was prepared in the Applicants' laboratory. It was raised against a synthetic peptide (here after referred as “JHS-7 peptide”) in rabbits. JHS-7 peptide contains 31 amino acids residues: “VLENSTKNSNDPAVFKDNPTEDVEYQCVADN” (SEQ ID NO: 3) and is flanked between two cation binding sites on the 5^{th} PECAM-1 (Ig)-like extracellular domain [18]. Anti-serum titer was tested by SDS-PAGE and JHS-7 Ab was purified by immunoaffinity column chromatography. In addition, a “scrambled peptide”, made of identical amino acid composition as JHS-7 peptide but different sequence (TEDVLVPQNKSDNKATNNAFPNVSYVDEEDC) (SEQ ID NO: 4) with no match of any known proteins or peptides, was also synthesized and served as a control of the JHS-7 peptide. The peptides were synthesized and purified at the Johns Hopkins University CORE servicing facility.

### Characterization of JHS-7 Antibody

Western blottin—The purified JHS-7 Ab was further characterized by 10% polyacrylamide gel (denatured) electrophoresis (SDS-PAGE) and Western immunoblotting. Equal amounts (25 μg) of total protein from HUVEC lysate and 50 ng of soluble recombinant PECAM-1 containing the extracellular domains only served as PECAM-1 antigen and subjected to gel electrophoresis. Three identical blots containing these two samples were probed by JHS-7 Ab, mAB-N and pAb-C respectively and comparison was made to verify the specificity of JHS-7 Ab.

In-direct immunofluorescent assays—In addition, indirect immunofluorescence was performed on a variety of PECAM-1 positive cells to detect native cellular PECAM-1. Cells grown on 8-well plastic chamber slides (Lab-Tek Chamber Slide® System, 177445, Nalge Nunc Int. Corp, Ill. USA) were fixed in 4% formaldehyde in PBS for 15 minutes at room temperature and subsequently permeabilized in 0.2% Triton X-100 in PBS for 15 minutes. Then the cells were incubated with JHS-7 Ab (1:50 dilution) for 1 hour followed by FITC-conjugated goat anti-rabbit IgG (1:300) for 1 hour at room temperature. An additional set of cells used for positive controls were incubated with mAb-N as primary anti-PECAM-1 antibody (1:250 dilution) and FITC-conjugated rabbit anti-mouse IgG (1:300). A Nikon fluorescent microscope was used to view the cells.

### Experimental Design

HUVECs were cultured in EGM™ supplied with 10% of fetal bovine serum (FBS, dialyzed) to early confluence. HUVECs (passage number 3-4) were pre-incubated with and without TNF-α (5 ng/mL, 4 hour), next the cells were subjected to various doses of the JHS-7 Ab, accompanied by the pAb-C (c-terminal anti-PECAM-1 antibody) and a rabbit IgG (both served as controls). In addition, HUVECs were also incubated with a various doses of the JHS-7 peptide and a scrambled peptide served as its control. The treatments were carried out in EGM™ supplied with 2% of FBS for 16 hours. This prolonged incubation time was to match the TEM findings since short-term incubation (1, 2 and 6 hours) with JHS-7 Ab and peptide failed to generate significant changes. All above-mentioned peptides and antibodies were tested endotoxin free with an endotoxin detection kit (Sigma E-TOXATE®). Cell viability was determined with trypan blue exclusion assay after the incubation and no cytotoxic effect was observed under the Applicants' experimental conditions. HUVEC monolayers, following the incubation, were subjected to the following assays simultaneously: 1) monocyte TEM assay; 2) [Ca²⁺]ᵢ measurement. Every experiment was repeated for three times or more each started with a new primary HUVEC pellet. Experimental results were calculated as average levels and plotted as % controls.

### U-937 Cell Trans-Endothelial Migration (TEM) Assay

A modified protocol of Transwell® assay described previously [28] was used. Briefly, HUVECs (1.0×10⁵ cells) were seeded on gelatin (0.2%)-coated upper wells (inserts) of the Transwell® design (12 mm diameter polycarbonate membrane with 3-μm pore size; Costar, Cambridge, Mass.) and grown for ˜3 days until they reached confluence. Following 4 hours of pre-activation with TNF-α, the monolayers of HUVECs were incubated with different doses of JHS-7 Ab and JHS-7 peptide and a variety of controls for 16 hours. Next, 1.0×10⁶ U-937 cells or freshly isolated human mononuclear cells were added in the upper well in fresh RPMI 1640 medium supplemented with 2% FBS and allowed to transmigrate through HUVEC monolayer for 12 hours. Analysis was carried out in quadruplicates. Finally, U-937 cells or mononuclear cells that transmigrated into the bottom wells were collected and counted.

### Measurement of Intracellular Free [Ca²⁺]ᵢ

[Ca²⁺]ᵢ measurement was conducted as described previously [29] with slight modification. Briefly, ˜4.5×10⁶ HUVECs treated with JHS-7 peptide and JHS-7 Ab for 16 hours were harvested by trypsinization according to standard procedures. HUVEC suspension were loaded with 1 μM of the fluorescent Ca²⁺ probe Fura-2/AM (Molecular Probes, Cat# F-1201, Eugene Oreg. USA) for 30 minutes at 37° C. with 5% CO₂ One third of cell suspension (˜1.5×10⁶) were added in triplicates in cuvettes and re-suspended in 2 ml of KRBH buffer (containing 136 mM NaCl, 4.8 mM KCl, 1.5 mM CaCl₂, 1.2 mM KH₂PO₄, 1.2 mM MgSO₄, 5 mM NaHCO₃ and 25 mM Hepes, pH 7.4). Fluorescence was recorded using a spectrofluorometer (Perkin Elmer LS-50B), with excitation and emission wavelengths of 340 and 505 nm, respectively. Following the baseline recording, extracellular thrombin (1 U/ml) was added to stimulate Ca²⁺ influx (from extracellular Ca²⁺) and release (from intracellular stores). To evaluate the effect of extracellular Ca²⁺, at one condition, EGTA (3 nM) was added ahead of thrombin to chalet the extracellular Ca²⁺.

### Statistics

Two-tailed T tests were performed. Results were expressed as the means±SD (standard deviation). Significant differences were considered when p value is less than 0.05.

### Example 1

### Identification of an Alternative PECAM-1 Transcript Missing the Entire 7^{Th} Exon which Encodes for the 5^{Th} Extracellular (Ig)-Like Domain

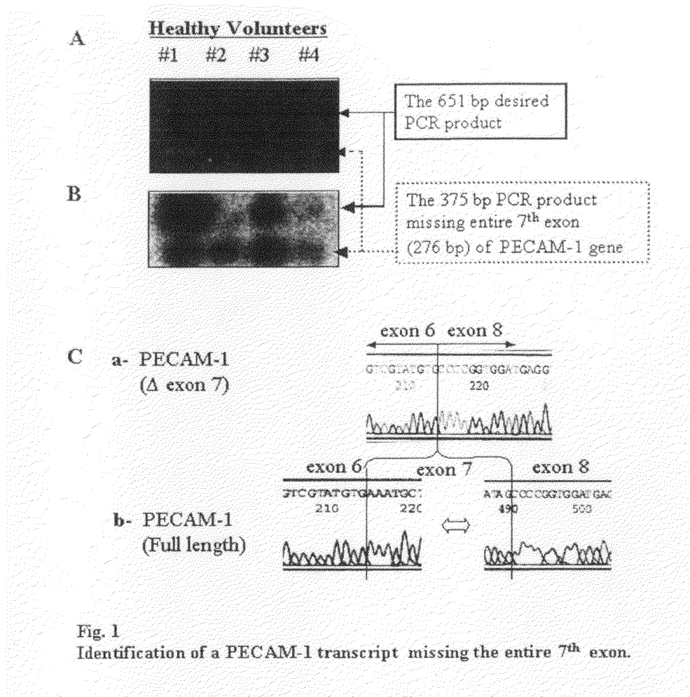

As shown in FIG. 1A, an additional and unprecedented lower PCR band with the size of 375 base pairs was generated in a PCR designed to amplify a 651 base pairs product covering several exons of the PECAM-1 coding sequence (cDNA templates were derived from total white blood cells of 4 healthy volunteers). The lower band was recognized and confirmed to be sequencing homology to PECAM-1 by a ³²P α-dCTP labeled cDNA probe during Southern hybridization (FIG. 1B). Next, TA cloned PCR products (both the upper and lower bands) were sequenced and a deletion of the entire 7^{th} exon (276 base pairs) in the lower band was confirmed (FIG. 1C).

### Example 2

### Characterization of JHS-7 Antibody

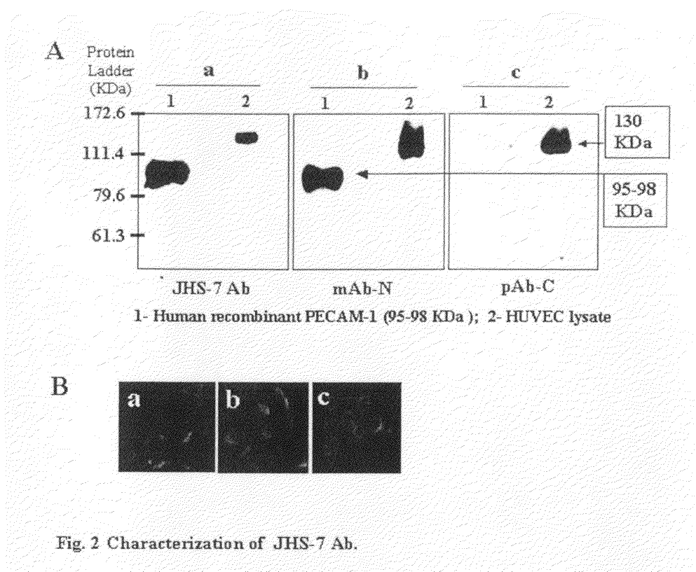

JHS-7 antibody recognized cellular PECAM-1 in HUVEC lysate by SDS-PAGE and Western blotting. Both JHS-7 Ab and the mAB-N (raised against the extracellular domains) (FIG. 2A a,b) recognized the standard PECAM-1 (soluble recombinant human PECAM-1, with size of 95˜98 KDa, containing extracellular domains only) as well as full length human PECAM-1 from HUVEC lysate (130 KDa) while pAb-C (against the cytoplasmic domains) failed to recognized soluble recombinant human PECAM-1 (FIG. 2A c). In addition, like mAb-N (data not shown), JHS-7 Ab was able to recognize native cellular PECAM-1 in HUVECs (FIG. 2B a), L-cells stably transfected with human PECAM-1 (a mouse fibroblast cell line previously transfected with human PECAM-1 in the Applicants' laboratory) (FIG. 2B b), and a promonocytic cell line, U-937 cells (FIG. 2B c) by indirect-immunofluorescent assay.

### Example 3

### JHS-7 Antibody and Peptide Attenuate the TEM of U-937 and Human Mononuclear Cells

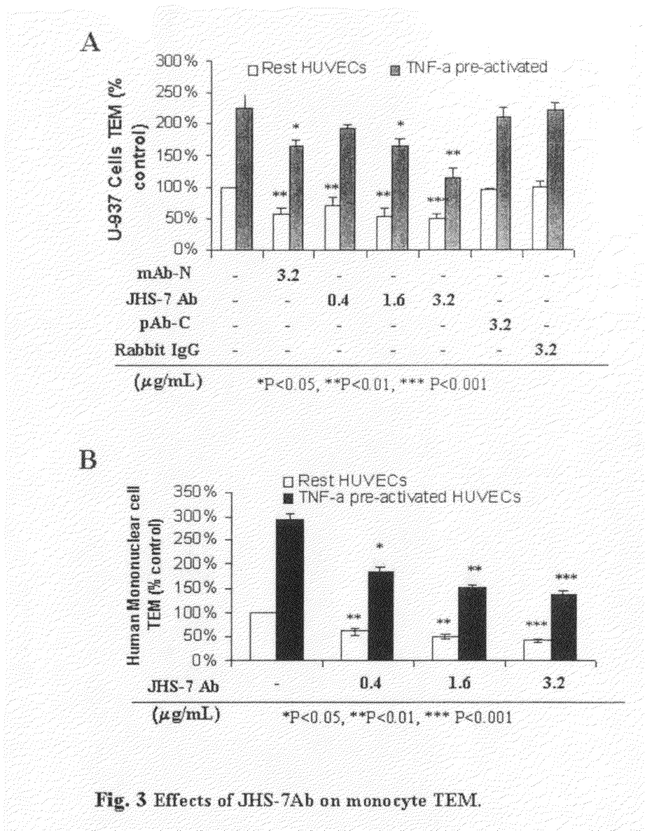

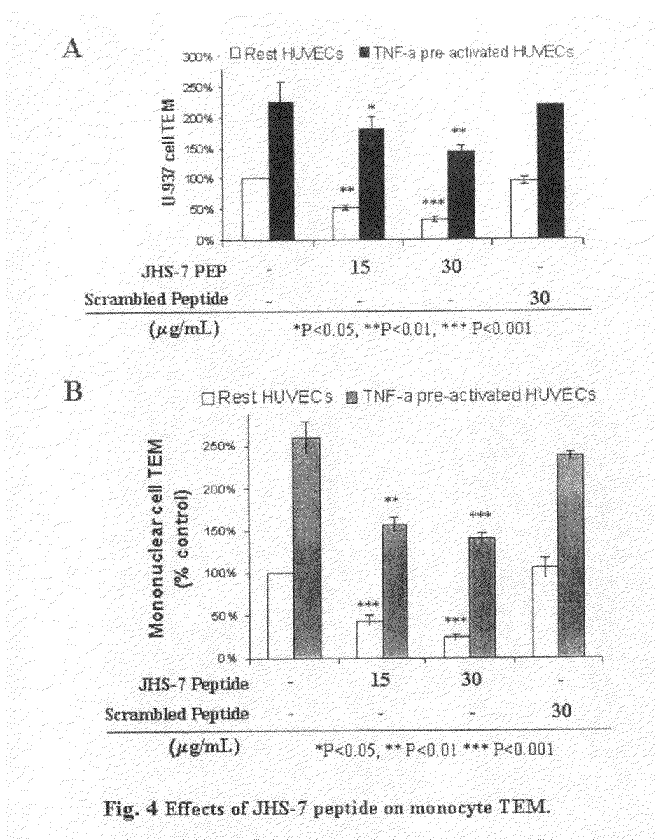

As shown in FIG. 3A, in HUVECs, TNF-α pre-activation led to markedly increase in the TEM of promonocytes (U-937). In both rest and TNF-α pre-activated HUVECs, various dilutions of JHS-7 Ab (0.4, 0.8 and 1.6 μg/mL), but not pAb-C (a control anti-PECAM-1 antibody against the C-terminal) (1.6 Ξg/mL), nor a rabbit IgG (1.6 μg/mL), significantly attenuated the trans-endothelial migration of U-937 cells down to ˜30.3±12.7% of the control level (FIG. 3A). The inhibitory effect of JHS-7 Ab on TEM was verified with freshly isolated human monocytes and similar results were obtained (FIG. 3B). Pre-incubation (16 hours) of HUVEC monolayers with JHS-7 peptide (15 and 30 μg/mL), but not scrambled peptide (15 and 30 μg/mL), attenuated TEM down to ˜23.0±5.5% of the control level in a concentration-dependent manner (FIG. 4A). Also the inhibitory effect of JHS-7 peptide on TEM was verified with freshly isolated human monocytes and similar results were obtained (FIG. 4B). Results from short-term incubations (1 hour, 2 hours and 6 hours) with both JHS-7 Ab and peptide were neither significant nor consistent although a trend of decreasing TEM was noted (data not shown).

### Example 4

### JHS-7 Peptide and AB Alter Intracellular Calcium Homeostasis

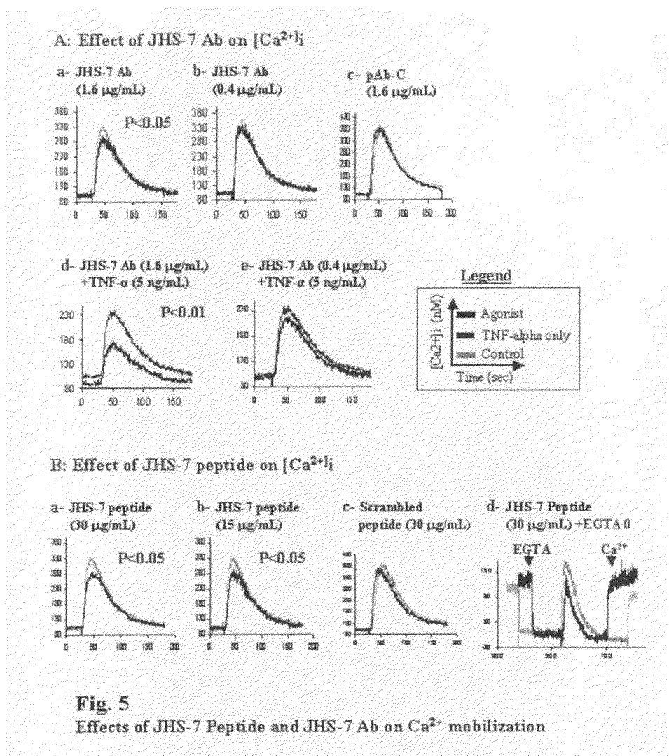

As seen in FIG. 5A, JHS-7 Ab exerted a concentration-dependent decrease in thrombin simulatable [Ca²⁺]ᵢ in rest HUVEC (FIG. 5A a,b) and more profoundly, in HUVECs pre-activated with 5 ng/mL of TNF-α for 4 hours (FIG. 5A d,e). No change was found with the rabbit IgG (FIG. 5A c). Similar effect was obtained with JHS-7 peptide and scrambled peptide showed no effect (FIG. 5B a,b,c). In addition, the effect of extracellular Ca²⁺ influx on the thrombin-regulated intracellular free [Ca²⁺]ᵢ was evaluated in JHS-7 peptide treated HUVECs. The addition of EGTA prior to thrombin (to bind extracellular Ca²⁺) failed to compromise the effect of JHS-7 peptide on decreasing the [Ca²⁺]ᵢ (FIG. 5Bd).

Short-term incubation with JHS-7 peptide and JHS-7 Ab (3, 15 minutes and 6 hours) failed to alter the level of baseline and thrombin stimulated [Ca²⁺]ᵢ significantly (data not shown).

According to what set forth above the Applicants' disclosure with a PECAM-1 peptide and corresponding anti-PECAM-1 antibody derived from the 5^{th} (Ig)-like domain of PECAM-1 demonstrated that this domain can have a functional role in mediating monocyte/leukocyte TEM and regulating intracellular calcium homeostasis.

In summary, an isolated polynucleotide coding for platelet endothelial cell adhesion molecule-1 (PECAM-1), and obtainable by amplifying cDNA from total human white blood cells by PCR; peptides encoding PECAM-1 5^{th} Ig-like domain; an antibody against one of the peptides; associated compositions methods and kits of parts.

The disclosures of each and every publication and reference cited herein are hereby incorporated herein by reference in their entirety.

The present disclosure has been explained with reference to specific embodiments. Other embodiments will be apparent to those of ordinary skill in the art in view of the foregoing description. The scope of protection of the present disclosure is defined by the appended claims.

### REFERENCES

[1] P. J. Newman, M. C. Berndt, J. Gorski, G. C. White, 2nd, S. Lyman, C. Paddock, and W. A. Muller, PECAM-1 (CD31) cloning and relation to adhesion molecules of the immunoglobulin gene superfamily, Science 247 (1990) 1219-1222.
[2] H. M. DeLisser, P. J. Newman, and S. M. Albelda, Platelet endothelial cell adhesion molecule (CD31), Curr Top Microbiol Immunol 184 (1993) 37-45.
[3] N. E. Kirschbaum, R. J. Gumina, and P. J. Newman, Organization of the gene for human platelet/endothelial cell adhesion molecule-1 shows alternatively spliced isoforms and a functionally complex cytoplasmic domain, Blood 84 (1994) 4028-4037.
[4] P. J. Newman, The biology of PECAM-1, J Clin Invest 99 (1997) 3-8.
[5] C. D. Buckley, R. Doyonnas, J. P. Newton, S. D. Blystone, E. J. Brown, S. M. Watt, and D. L. Simmons, Identification of alpha v beta 3 as a heterotypic ligand for CD31/PECAM-1, J Cell Sci 109 (Pt 2) (1996) 437-445.
[6] D. Varon, D. E. Jackson, B. Shenkman, R. Dardik, I. Tamarin, N. Savion, and P. J. Newman, Platelet/endothelial cell adhesion molecule-1 serves as a costimulatory agonist receptor that modulates integrin-dependent adhesion and aggregation of human platelets, Blood 91 (1998) 500-507.
[7] W. A. Muller, S. A. Weigi, X. Deng, and D. M. Phillips, PECAM-1 is required for transendothelial migration of leukocytes, J Exp Med 178 (1993) 449-460.
[8] K. E. Noble, R. G. Wickremasinghe, C. DeCornet, P. Panayiotidis, and K. L. Yong, Monocytes stimulate expression of the Bcl-2 family member, A1, in endothelial cells and confer protection against apoptosis, J Immunol 162 (1999) 1376-1383.
[9] C. Gao, W. Sun, M. Christofidou-Solomidou, M. Sawada, D. K. Newman, C. Bergom, S. M. Albelda, S. Matsuyama, and P. J. Newman, PECAM-1 functions as a specific and potent inhibitor of mitochondrial-dependent apoptosis, Blood (2003).
[10] W. A. Muller, The role of PECAM-1 (CD31) in leukocyte emigration: studies in vitro and in vivo, J Leukoc Biol 57 (1995) 523-528.
[11] M. W. Wakelin, M. J. Sanz, A. Dewar, S. M. Albelda, S. W. Larkin, N. Boughton-Smith, T. J. Williams, and S. Nourshargh, An anti-platelet-endothelial cell adhesion molecule-1 antibody inhibits leukocyte extravasation from mesenteric microvessels in vivo by blocking the passage through the basement membrane, J Exp Med 184 (1996) 229-239.
[12] S. Bogen, J. Pak, M. Garifallou, X. Deng, and W. A. Muller, Monoclonal antibody to murine PECAM-1 (CD31) blocks acute inflammation in vivo, J Exp Med 179 (1994) 1059-1064.
[13] F. Liao, H. K. Huynh, A. Eiroa, T. Greene, E. Polizzi, and W. A. Muller, Migration of monocytes across endothelium and passage through extracellular matrix involve separate molecular domains of PECAM-1, J Exp Med 182 (1995) 1337-1343.
[14] R. D. Thompson, K. E. Noble, K. Y. Larbi, A. Dewar, G. S. Duncan, T. W. Mak, and S. Nourshargh, Platelet-endothelial cell adhesion molecule-1 (PECAM-1)-deficient mice demonstrate a transient and cytokine-specific role for PECAM-1 in leukocyte migration through the perivascular basement membrane, Blood 97 (2001) 1854-1860.
[15] Q. H. Sun, H. M. DeLisser, M. M. Zukowski, C. Paddock, S. M. Albelda, and P. J. Newman, Individually distinct Ig homology domains in PECAM-1 regulate homophilic binding and modulate receptor affinity, J Biol Chem 271 (1996) 11090-11098.
[16] J. P. Newton, C. D. Buckley, E. Y. Jones, and D. L. Simmons, Residues on both faces of the first immunoglobulin fold contribute to homophilic binding sites of PECAM-1/CD31, J Biol Chem 272 (1997) 20555-20563.
[17] F. Liao, J. Ali, T. Greene, and W. A. Muller, Soluble domain 1 of platelet-endothelial cell adhesion molecule (PECAM) is sufficient to block transendothelial migration in vitro and in vivo, J Exp Med 185 (1997) 1349-1357.
[18] D. E. Jackson, R. O. Loo, M. T. Holyst, and P. J. Newman, Identification and characterization of functional cation coordination sites in platelet endothelial cell adhesion molecule-1, Biochemistry 36 (1997) 9395-9404.
[19]1. Gurubhagavatula, Y. Amrani, D. Pratico, F. L. Ruberg, S. M. Albelda, and R. A. Panettieri, Jr., Engagement of human PECAM-1 (CD31) on human endothelial cells increases intracellular calcium ion concentration and stimulates prostacyclin release, J Clin Invest 101 (1998) 212-222.
[20] C. D. O'Brien, G. Ji, Y. X. Wang, J. Sun, V. P. Krymskaya, F. L. Ruberg, M. I. Kotlikoff, and S. M. Albelda, PECAM-1 (CD31) engagement activates a phosphoinositide-independent, nonspecific cation channel in endothelial cells, Faseb J 15 (2001) 1257-1260.
[21] N. Ilan, S. Mahooti, D. L. Rimm, and J. A. Madri, PECAM-1 (CD31) functions as a reservoir for and a modulator of tyrosine-phosphorylated beta-catenin, J Cell Sci 112 Pt 18 (1999) 3005-3014.
[22] N. Ilan, L. Cheung, E. Pinter, and J. A. Madri, Platelet-endothelial cell adhesion molecule-1 (CD31), a scaffolding molecule for selected catenin family members whose binding is mediated by different tyrosine and serine/threonine phosphorylation, J Biol Chem 275 (2000) 21435-21443.
[23] N. Ilan, L. Cheung, S. Miller, A. Mohsenin, A. Tucker, and J. A. Madri, Pecam-1 is a modulator of stat family member phosphorylation and localization: lessons from a transgenic mouse, Dev Biol 232 (2001) 219-232.
[24] N. Sheibani, C. M. Sorenson, and W. A. Frazier, Tissue specific expression of alternatively spliced murine PECAM-1 isoforms, Dev Dyn 214 (1999) 44-54.
[25] F. Aroca, W. Renaud, C. Bartoli, C. Bouvier-Labit, and D. Figarella-Branger, Expression of PECAM-1/CD31 isoforms in human brain gliomas, J Neurooncol 43 (1999) 19-25.
[26] Y. Wang, and N. Sheibani, Expression pattern of alternatively spliced PECAM-1 isoforms in hematopoietic cells and platelets, J Cell Biochem 87 (2002) 424-438.
[27] J. M. Wang, A. Sica, G. Peri, S. Walter, I. M. Padura, P. Libby, M. Ceska, I. Lindley, F. Colotta, and A. Mantovani, Expression of monocyte chemotactic protein and interleukin-8 by cytokine-activated human vascular smooth muscle cells, Arterioscler Thromb 11 (1991) 1166-1174.
[28] D. Y. Jo, S. Rafii, T. Hamada, and M. A. Moore, Chemotaxis of primitive hematopoietic cells in response to stromal cell-derived factor-1, J Clin Invest 105 (2000) 101-111.
[29] G. Li, H. Hidaka, and C. B. Wollheim, Inhibition of voltage-gated Ca²⁺ channels and insulin secretion in HIT cells by the Ca²⁺/calmodulin-dependent protein kinase II inhibitor KN-62: comparison with antagonists of calmodulin and L-type Ca²⁺ channels, Mol Pharmacol 42 (1992) 489-488.
[30] A. Goldberger, K. A. Middleton, J. A. Oliver, C. Paddock, H. C. Yan, H. M. DeLisser, S. M. Albelda, and P. J. Newman, Biosynthesis and processing of the cell adhesion molecule PECAM-1 includes production of a soluble form, J Biol Chem 269 (1994) 17183-17191.
[31] V. L. Serebruany, S. R. Murugesan, A. Pothula, H. Semaan, and P. A. Gurbel, Soluble PECAM-1, but not P-selectin, nor osteonectin identify acute myocardial infarction in patients presenting with chest pain, Cardiology 91 (1999) 50-55.
[32] A. A. Vaporciyan, H. M. DeLisser, H. C. Yan, Mendiguren, II, S. R. Thom, M. L. Jones, P. A. Ward, and S. M. Albelda, Involvement of platelet-endothelial cell adhesion molecule-1 in neutrophil recruitment in vivo, Science 262 (1993) 1580-1582.
[33] J. Sun, J. Williams, H. C. Yan, K. M. Amin, S. M. Albelda, and H. M. DeLisser, Platelet endothelial cell adhesion molecule-1 (PECAM-1) homophilic adhesion is mediated by immunoglobulin-like domains 1 and 2 and depends on the cytoplasmic domain and the level of surface expression, J Biol Chem 271 (1996) 18561-18570.
[34] R. D. Thompson, M. W. Wakelin, K. Y. Larbi, A. Dewar, G. Asimakopoulos, M. A. Horton, M. T. Nakada, and S. Nourshargh, Divergent effects of platelet-endothelial cell adhesion molecule-1 and beta 3 integrin blockade on leukocyte transmigration in vivo, J Immunol 165 (2000) 426-434.
[35] L. Piali, P. Hammel, C. Uherek, F. Bachmann, R. H. Gisler, D. Dunon, and B. A. Imhof, CD31/PECAM-1 is a ligand for alpha v beta 3 integrin involved in adhesion of leukocytes to endothelium, J Cell Biol 130 (1995) 451-460.
[36] K. Kielbassa-Schnepp, A. Strey, A. Janning, L. Missiaen, B. Nilius, and V. Gerke, Endothelial intracellular Ca²⁺ release following monocyte adhesion is required for the transendothelial migration of monocytes, Cell Calcium 30 (2001) 29-40.
[37] M. Cicmil, J. M. Thomas, T. Sage, F. A. Barry, M. Leduc, C. Bon, and J. M. Gibbins, Collagen, convulxin, and thrombin stimulate aggregation-independent tyrosine phosphorylation of CD31 in platelets. Evidence for the involvement of Src family kinases, J Biol Chem 275 (2000) 27339-27347.

1.-6. (canceled)

7. A method to regulate intracellular calcium homeostasis of a human cell, the method comprising administering to the human cell an effective amount of a peptide having amino acid sequence of SEQ ID NO: 3 and/or an effective amount of an antibody which bonds to an isolated peptide of SEQ ID NO: 3.

8. Method for inhibiting for inhibiting PECAM-1 dependent trans-endothelial migration of a monocyte, the method comprising administering to the monocyte an effective amount of a peptide having amino acid sequence of SEQ ID NO: 3 and/or an effective amount of an antibody which bonds to an isolated peptide of SEQ ID NO: 3.

9. A kit of parts for the regulation of intracellular calcium homeostasis of a human cell, the kit comprising a peptide comprising the sequence SEQ ID NO: 3; and an antibody which bonds to an isolated peptide having the amino acid sequence of SEQ ID NO: 3 the peptide and the antibody to be administered in an effective amount to the human cell thereby regulating the intracellular calcium homeostasis of the human cell.

10. The kit of part of claim 9, the kit further comprising a second peptide comprising the sequence SEQ ID NO:4 the second peptide to be administered in combination with the first peptide and the antibody as a negative control wherein administration of the effective amount of the first peptide and the antibody regulate the intracellular calcium homeostasis of the human cell.

11. A kit of parts for inhibiting PECAM-1 dependent trans-endothelial migration of a monocyte, the kit comprising a first peptide comprising the sequence SEQ ID NO: 3; and an antibody which bonds to an isolated peptide having the amino acid sequence of SEQ ID NO: 3 the first peptide and the antibody to be administered in an effective amount to a monocyte thereby inhibiting trans-endothelial migration of the monocyte the trans-endothelial migration dependent on platelet endothelial cell adhesion molecule-1.

12. The kit of part of claim 11, the kit further comprising a second peptide comprising the sequence SEQ ID NO: 4, the second peptide to be administered in combination with the first peptide and the antibody as a negative control wherein administration of the effective amount of the first peptide and the antibody inhibit trans-endothelial migration of the monocyte the trans-endothelial migration dependent on platelet endothelial cell adhesion molecule-1.
